(19) United States
(12) Patent Application Publication
(10) Pub. No.: US 2006/0025698 A1
(43) Pub. Date: Feb. 2, 2006

(54) Method and device for displaying living body information

(75) Inventors: Tsuyoshi Nakagawa, Aichi-gun (JP); Taiji Kawachi, Kariya-city (JP); Akiko Ito, Nagoya-city (JP)

Correspondence Address: POSZ LAW GROUP, PLC, 12040 SOUTH LAKES DRIVE, SUITE 101, RESTON, VA, 20191, US

(73) Assignee: DENSO CORPORATION

(21) Appl. No.: 11/159,266

(22) Filed: Jun. 23, 2005

(30) Foreign Application Priority Data
Jul. 29, 2004 (JP) 2004-222241

(51) Int. Cl. A61B 5/04

(52) U.S. Cl. 600/513

(57) ABSTRACT

At data updating timing, a two-dimensional coordinate system is shown on a display screen based on a display condition that is separately set; further, a region of a physical condition is shown to indicate a correspondence relationship between a driver's physical condition and points on the two-dimensional coordinate system. A heartbeat rate analyzing process and a blood pressure analyzing process produce, as a result, detection data P(i) including a heartbeat rate HR and a blood pressure BP. The detection data P(i) is shown on the two-dimensional coordinate system with an X-axis of a heartbeat rate HR and a Y-axis of a blood pressure BP. Further, passed detection data P(i−1)˜P(i−k) numbering k are additionally shown so that older data is brighter in a color tone.

### CROSS REFERENCE TO RELATED APPLICATIONS

This application is based on and incorporates herein by reference Japanese Patent Application No. 2004-222241 filed on Jul. 29, 2004.

### FIELD OF THE INVENTION

The present invention relates to a method and a device for displaying two kinds of living body information relating to a subject.

### BACKGROUND OF THE INVENTION

There are devices measuring living body information of subjects that includes a blood pressure or a heartbeat rate, in medical sites. These devices are adapted to devices used by non-professional people other than medical professionals in ordinal healthcares without being limited to the medical sites. For instance, the devices are further adapted to devices used for assisting operating by drivers or determining fatigue degrees of drivers.

A device used in the medical sites includes a blood pressure meter, a heartbeat rate meter, or a blood oxygen level meter. The device displays in real time numerical values of blood pressures or heartbeat rates of subject patients or displays graphs representing time-series variations with respect to each kind of living body information.

A device used in the ordinal healthcares includes a wrist-watch-shaped measuring device that detects electrocardiograph waves and pulse waves. This measuring device thereby obtains a blood pressure from a transmission time of the pulse waves computed from the detection result and further displays as numeric values or graphs the blood pressure obtained (refer to Patent Document 1).

A device used in assisting operating by drivers detects variations in a heartbeat rate or a heartbeat interval as living body information of a driver to then execute a process for determining a state of the driver when the detection result has abnormality (refer to Patent Document 2). Further, a device determines a fatigue degree to then display the determination result or execute a vehicle control based on the determination result (refer to Patent Document 3). Here, the fatigue degree is determined based on a heartbeat rate and a time-series transition tendency of a distributional region where detection results are associated in a two-dimensional coordinate system.
Patent Document 1: JP-H4-200439A (U.S. Pat. No. 5,316,008)
Patent Document 2: JP-2002-74599 A
Patent Document 3: JP-2002-65650 A

The device used in medical sites or the device in Patent Document 1 displays individual kinds of living body information to indicate variations of the individual kinds of living body information. However, non-professional people having no expert knowledge cannot easily understand nor determine what the variations mean.

Further, the devices in Patent Documents 1, 2 determine the states of the drivers based on the living body information detected. The detection results are communicated or used for various controls so that the subjects can understand reaching the states determined. However, they cannot understand in real time own states that vary momentarily.

### SUMMARY OF THE INVENTION

It is an object of the present invention to provide a displaying method or a displaying device that enables variations of living body information to be understood in real time and meaning of the variations to be quickly and properly determined.

To achieve the above object, as a first aspect of the present invention, a living body information displaying method is provided with steps of the following. A first step is associating detection results of two kinds of living body information, which are obtained with respect to a subject, with a point on a two-dimensional coordinate system that is set on a display screen. A second step is displaying on the display screen the detection results along with previously obtained detection results of the two kinds of living body information and a region which indicates a correspondence relationship between a point on the two-dimensional coordinate system and a state of the subject.

Under this structure, the living body information of the subject can be displayed as points on a two-dimensional coordinate system with the region indicating the corresponding state of the subject to be thereby easily understood without any professional medical knowledge. Further, additionally displaying the previously obtained detection results can indicate not only the present state but also variations in the state in real time.

To achieve the above object, as a second aspect of the present invention, a living body information displaying device is provided with the following. A detecting unit is included for obtaining detection results of at least two kinds of living body information based on a pulse wave signal and an electrocardiograph signal of a subject. A detection result displaying unit is included for associating the detection results with a point on a two-dimensional coordinate system that is set on a display screen to thereby display the detection results along with previously obtained detection results on the display screen. A region displaying unit is included for displaying a plurality of regions that indicate correspondence relationships between points of the two-dimensional coordinate system and states of the subject on the display screen while overlapping the regions on detection results displayed by the detection result displaying unit.

In other words, this device achieves the displaying method of the first aspect of the present invention to thereby obtain the same effect as the first aspect.

### BRIEF DESCRIPTION OF THE DRAWINGS

The above and other objects, features, and advantages of the present invention will become more apparent from the following detailed description made with reference to the accompanying drawings. In the drawings:

FIG. 1 is a block diagram showing a structure of a physical condition monitor system according to an embodiment of the present invention;

FIG. 2 is a view explaining layouts of components of a physical condition monitor system;

FIG. 3A is a view showing an appearance of an electrocardiograph sensor;

FIG. 3B is a view showing an appearance of a pulse wave sensor;

FIG. 4 is a flowchart diagram explaining an electrocardiograph signal analyzing process;

FIG. 5 is a flowchart diagram explaining a pulse wave signal analyzing process;

FIG. 6 is a flowchart diagram explaining a heartbeat rate analyzing process;

FIG. 7 is a flowchart diagram explaining a blood pressure analyzing process;

FIG. 8 is a flowchart diagram explaining a display condition setting process;

FIG. 9 is a flowchart diagram explaining a matrix displaying process;

FIG. 10 is a flowchart diagram explaining a blood pressure abnormality determining process;

FIG. 11 is a flowchart diagram explaining a drowsiness/distraction determining process;

FIG. 12 is graphs explaining an electrocardiograph signal analyzing process;

FIG. 13 is a graph explaining a computing method for pulse wave transmission time;

FIG. 14 is a graph showing a regional display in a matrix display of a blood pressure and a heartbeat rate;

FIG. 15 is a graph showing a determination example in FIG. 14;

FIG. 16 is a graph showing an absolute display in a matrix display of detection data and record data;

FIG. 17 is a graph showing a relative display in a matrix display of detection data and record data;

FIG. 18 is a graph including a maximum blood pressure and a minimum blood pressure in addition to an absolute display in a matrix display of detection data and record data;

FIG. 19 is a graph showing a regional display in a matrix display of an autonomic nerve activity amount;

FIG. 20 is graphs showing a correspondence relationship between a matrix display of a blood pressure and a heartbeat rate and a matrix display of an autonomic nerve activity amount; and

FIG. 21 is a view showing another example showing a layout of a pulse wave sensor.

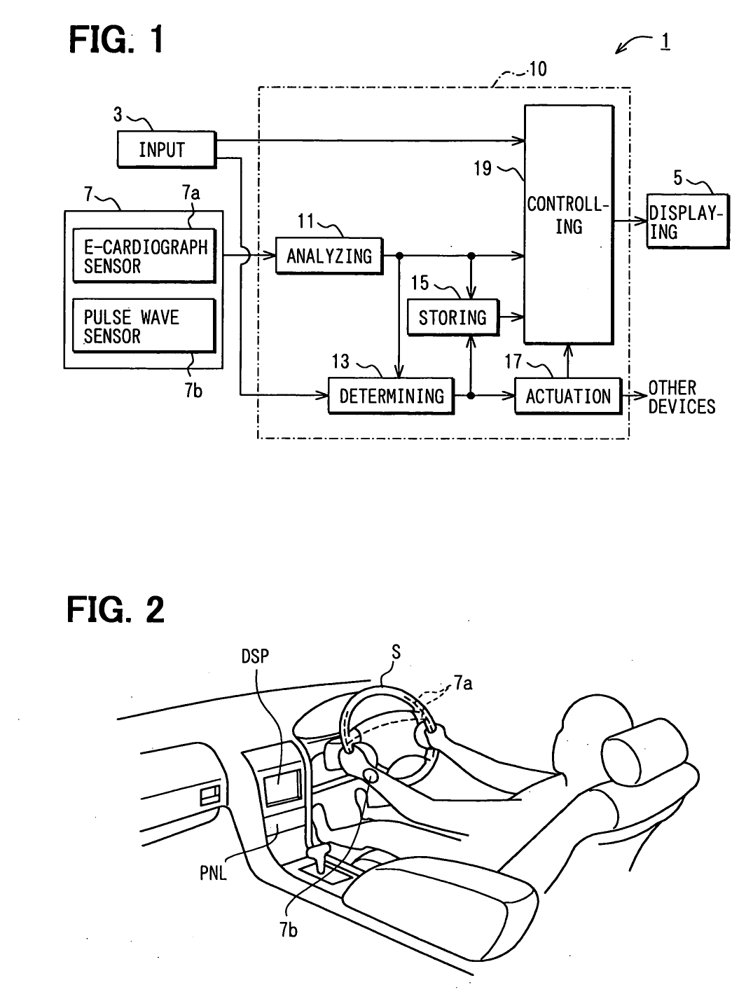

### DETAILED DESCRIPTION OF THE PREFERRED EMBODIMENTS

A physical condition monitor system 1 according to an embodiment of the present invention monitors a physical condition of a driver operating a vehicle. The system 1 includes the following: an input unit 3 for inputting various instructions or data; a displaying unit 5 for displaying an operation procedure, a data input window, detection results, determination results, or the like; a signal measuring unit 7 for detecting, of the driver, electrocardiograph signals and pulse wave signals using an electrocardiograph sensor 7a and a pulse wave sensor 7b; a signal analyzing unit 11 for analyzing the electrocardiograph signals and pulse wave signals from the signal measuring unit 7 to thereby obtain living body information such as a blood pressure, a heartbeat rate, or an autonomic nerve activity amount; a state determining unit 13 for determining a physical condition or state of the driver based on the living body information obtained by the signal analyzing unit 11; a data storing unit 15 for storing the analysis results by the signal analyzing unit 11, the determination results by the state determining unit 13, determination conditions used when the state determining unit 13 determines, display conditions used when the displaying unit 5 displays, or the like; an actuation unit 17 for executing various controls based on the determination results by the state determining unit 13; and a display controlling unit 19 for causing the displaying unit 5 to display the living body information obtained by the signal analyzing unit 11, or various data stored by the data storing unit 15 based on the instructions from the input unit 3 or the actuation unit 17.

Here, in detail, the input unit 3 and the displaying unit 5 include an input panel PNL and a display DSP in a navigation device mounted in the vehicle, respectively. Further, from the input unit 3, at least a display form and kinds of the living body information to be displayed can be designated. The display form includes a matrix display or a trend display, or an absolute display or a relative display.

Living body information to be displayed can be selected from either a pair of a blood pressure and a heartbeat rate or an autonomic nerve (a pair of a sympathetic nerve activity and a parasympathetic nerve activity). Further, values of the two kinds of living body information can be displayed as either a matrix display or a trend display. In the matrix display, the values of the two kinds of living body information are displayed while being associating with points in the two-dimensional coordinate system. In the trend display, the values of each of the two kinds of living body information are displayed as a line chart while each kind of living body information being a Y-axis (ordinate axis) and time being as an X-axis (abscissa axis). Furthermore, in the matrix display, the values of the two kinds of living body information to be displayed can be displayed in either an absolute display or a relative display. Further, in the matrix display, the values of the two kinds of living body information can be also accompanied by maximum values or minimum values.

Further, from the input unit 3, various conditions can be designated. These conditions include: a display condition used when a blood pressure and a heartbeat rate are displayed as a matrix display; and a determination condition used when a blood pressure abnormality determining process is executed by the state determining unit 13 or when a drowsiness/distraction determining process is executed.

In detail, the display condition includes: ranges (upper limits, lower limits) of a blood pressure and a heartbeat rate in a display screen; conditions for setting coordinate centers C that are sets of coordinates located in a center of a display screen such as an initial blood pressure BPr, and an initial heartbeat rate HRr; or time data such as a data update cycle Mt for updating a display to the latest data, a record data display time Ht representing a holding time for holding displaying of record data (or passed data).

The determination condition in the blood pressure abnormality determination includes: a determination cycle T1 for blood pressure abnormality; a high blood pressure threshold value BPU; and a low blood pressure threshold value BPD. The high blood pressure threshold value BPU and the low blood pressure threshold value BPD define a normal blood pressure area. The determination condition in the drowsiness/distraction determination includes: a determination cycle T2 for drowsiness/distraction; an upper limit threshold value RBPU1, RBPU2 for a blood pressure ratio; a lower limit threshold value RBPD1, RBPD2 for a blood pressure ratio; an upper limit threshold value RHRU1, RHRU2 for a heartbeat rate ratio; and a lower limit threshold value RHRD1, RHRD2 for a heartbeat rate ratio. Here, the blood pressure ratio is a ratio of an average blood pressure to a predetermined blood pressure reference value within the determination cycle T2. The upper limit threshold value RHRU1, RHRU2 and the lower limit threshold value RHRD1, RHRD2 define a normal area of a heartbeat rate ratio.

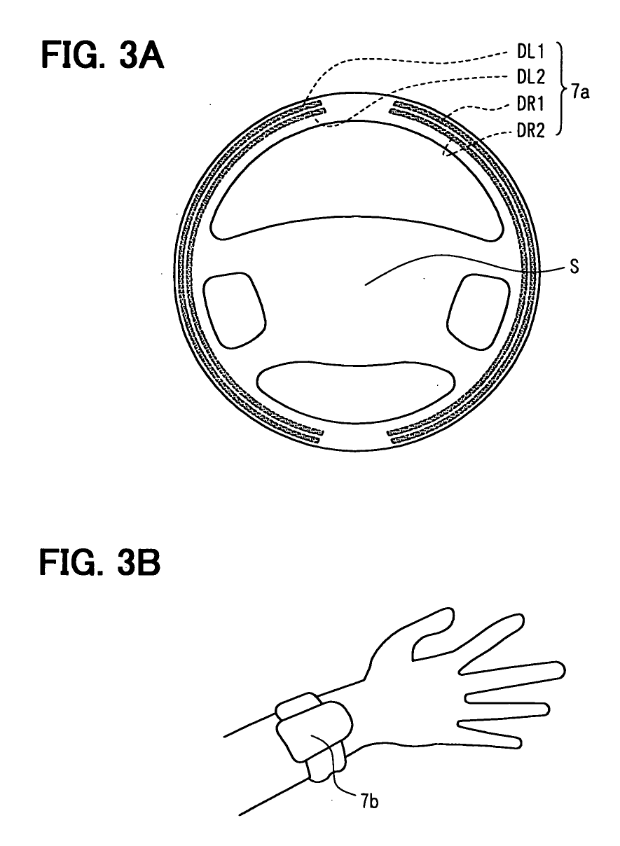

Next, The electrocardiograph sensor 7a detects electrocardiograph signals using detection electrodes of two pairs of electrodes DR1, DR2, DL1, DL2 that are embedded within portions of a steering wheel S that the driver holds using the right hand and the left hand, as shown in FIG. 2 and FIG. 3A. The pulse wave sensor 7b is a wrist-watch-shaped sensor that is attached to a wrist of the driver as shown in FIG. 3B. The pulse sensor 7b is a known optical plethysmogram (PTG) meter that optically detects variations of a blood vessel volume.

The signal measuring unit 7 samples with predetermined intervals (here, 10 ms) electrocardiograph signals from the electrocardiograph sensor 7a and pulse signals from the pulse wave sensor 7b to then give them to the signal analyzing unit 11.

The signal analyzing unit 11, the state determining unit 13, the data storing unit 15, the actuation unit 17, and the display controlling unit 19 are practically included in an electronic control unit (ECU) 10 mainly constituted by a micro-computer including a CPU, a ROM, and a RAM. In particular, the signal analyzing unit 11, the state determining unit 13, the actuation unit 17, and the display controlling unit 19 are achieved as process executed by the ECU 10, while the data storing unit 15 is constructed on memory storage such as the RAM.

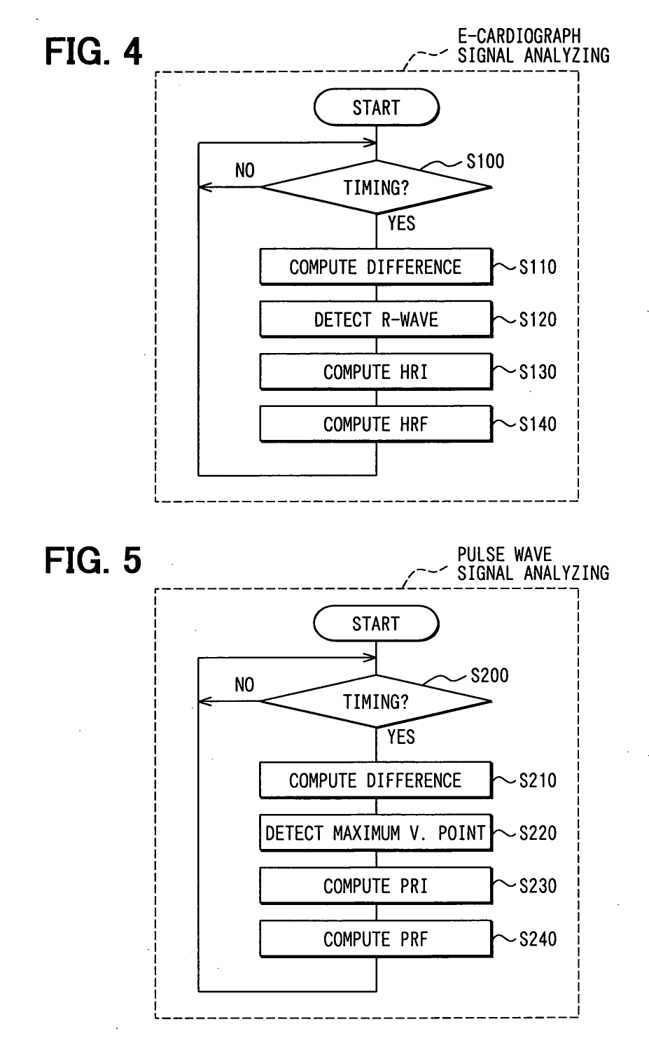

Hereinbelow, process executed by the CPU will be explained. At first, the process corresponding to the signal analyzing unit 11 will be explained with reference to FIGS. 4 to 7. The process corresponding to the signal analyzing unit 11 includes an electrocardiograph signal analyzing process, a pulse signal analyzing process, a heartbeat rate analyzing process, or a blood pressure analyzing process.

The electrocardiograph signal analyzing process and the pulse signal analyzing process start when electric power is supplied to the physical condition monitor system 1. The heartbeat rate analyzing process and the blood pressure analyzing process start each time analysis results are obtained from the electrocardiograph signal analyzing process and the pulse signal analyzing process.

In the electrocardiograph signal analyzing process, at first, at Step S100, it is determined whether it is an analyzing timing or not. Here, the analyzing timing is set in 1-second intervals.

When it is an analyzing timing, a difference process (computing difference) takes place with respect to, as an analyzing target, sampling values (hereinafter, referred to only “electrocardiograph signals) of electrocardiograph signals for passed 10 seconds at Step S110. In this difference process, an electrocardiograph signal sampled at time (t) is X(t); an electrocardiograph signal sampled second most previously closest to the signal sampled at time (t) is X(t−2). An electrocardiograph difference signal Y(t) is obtained from Equation (1).
Y(t)=X(t)−X(t−2) (1)

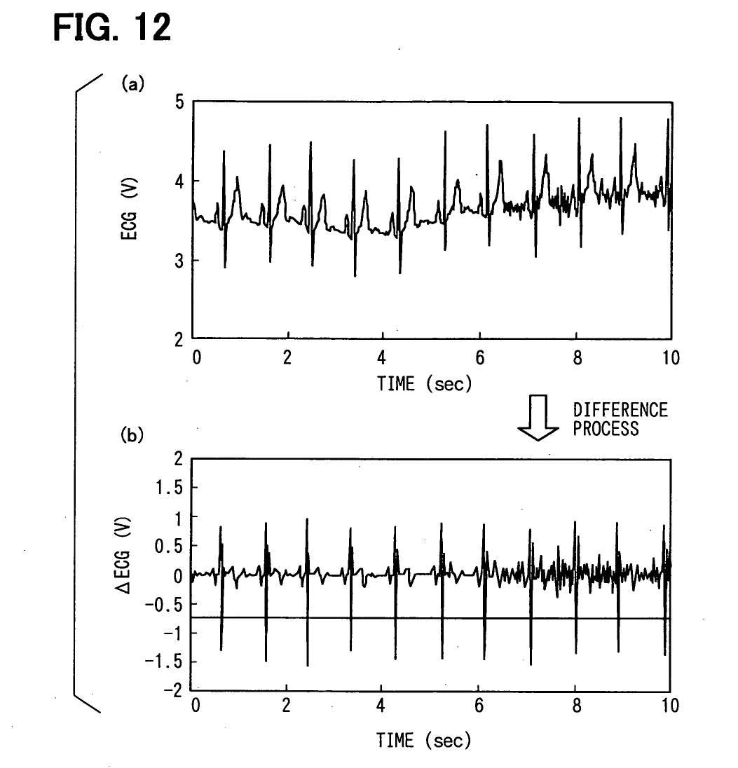

An example of electrocardiograph signals is shown in FIG. 12A; corresponding electrocardiograph difference signals obtained by subjecting the electrocardiograph signals to the difference process are shown in FIG. 12B.

The electrocardiograph difference signals Y(t) for 10 seconds are computed in block at each analyzing timing; however, the difference signals can be computed each time an electrocardiograph signal is supplied from the signal measuring unit 7.

At Step S120, an R-wave peak that is larger than a predetermined threshold value is detected from the electrocardiograph difference signals obtained as a result from the difference process. At Step S130, an average value PPlav is obtained as an average of peak intervals PRI between the detected R-wave peaks. A heartbeat rate HRI is computed as an average movement of ten average values PPIav computed for passed 10 seconds. The electrocardiograph signals of the analyzing target excluding the R-wave peaks detected at Step S120 are set to zero and then are subjected to an FFT (Fast Fourier Transform) process to thereby obtain an R-wave peak repetition frequency FRP. At Step S140, a heartbeat rate HRF is computed as an inverse of an average movement of ten frequencies FRP computed for passed 10 seconds. The sequence then returns to Step S100.

Namely, executing the electrocardiograph signal analyzing process causes with respect to the analyzing timing (1 second) a heartbeat rate HRI obtained from intervals of the R-wave peaks of electrocardiograph signals, and a heartbeat rate HRI obtained by subjecting the electrocardiograph signals to the FFT process.

Next, in the pulse signal analyzing process, as shown in FIG. 5, at first, at Step S200, it is determined whether it is an analyzing timing or not. Here, the analyzing timing is set in 1-second intervals similarly to that of the electrocardiograph signals.

When it is an analyzing timing, a difference process takes place with respect to, as an analyzing target, sampling values (hereinafter, referred to only “pulse wave signals”) of pulse wave signals for passed 10 seconds at Step S210, similarly to the electrocardiograph signals. In this difference process, a maximum varying point is detected as a peak larger than a predetermined threshold value from the pulse wave difference signals at Step S220.

Then, an average value Plav is obtained as an average of peak intervals between the detected maximum varying points. At Step S230, a heartbeat rate PRI is computed as an average movement of ten average values Plav computed for passed 10 seconds. The pulse wave signals of the analyzing target are subjected to an FFT process to thereby obtain a pulse wave repetition frequency FPI. At Step S240, a heartbeat rate PRF is computed as an inverse of an average movement of ten frequencies FPI computed for passed 10 seconds. The sequence then returns to Step S200.

Namely, executing the pulse wave signal analyzing process causes with respect to the analyzing timing (1 second) a heartbeat rate PRI obtained from intervals of the maximum varying points of pulse wave signals, and a heartbeat rate PRF obtained by subjecting the pulse wave signals to the FFT process.

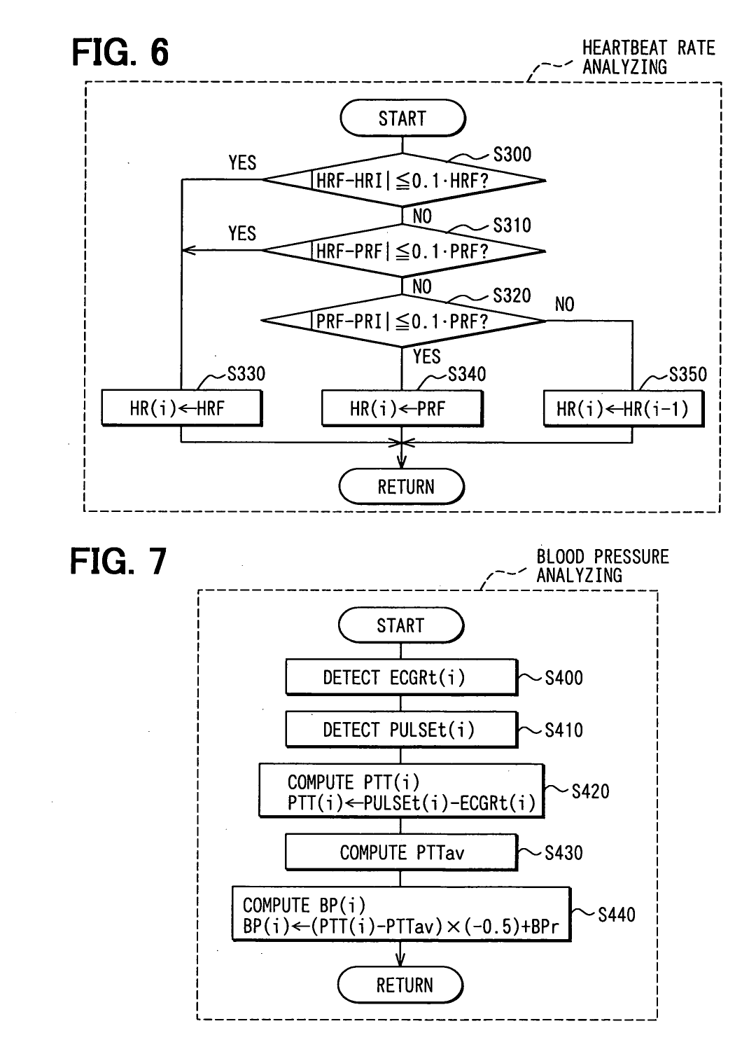

Next, in the heartbeat rate analyzing process, as shown in FIG. 6, at first, at Step S300 it is determined whether an absolute value of a difference between the heartbeat rate HRF obtained by subjecting the electrocardiograph signals to the FFT process and the heartbeat rate HRI obtained from the R-wave peak intervals of the electrocardiograph signals is 10% or less of the heartbeat rate HRF. When the determination at Step S300 is negated, it is then determined whether an absolute value of a difference between the heartbeat rate HRF obtained by subjecting the electrocardiograph signals to the FFT process and the heartbeat rate PRF obtained by subjecting the pulse wave signals to the FFT process is 10% or less of the heartbeat rate PRF at Step S310.

When either determination at Step S300 or determination at Step S310 is affirmed, the heartbeat rate HRF obtained by subjecting the electrocardiograph signals to the FFT process is set as a detection heartbeat rate HR(i) at Step S330. This process then ends. Here, HR(i) is the latest detection value; HR(i−k) is a detection value that is detected k-th previously closest to HR(i) (e.g., HR(i−1) is detected immediate-previously with respect to HR(i), HR(i−2) is detected second previously closest to HR(i)). This abbreviating rule regarding order is applied to others below.

In contrast, when the determination at Step S310 is negated, it is determined whether an absolute value of a difference between the heartbeat rate PRF obtained by subjecting the pulse wave signals to the FFT process and the heartbeat rate PRI obtained from the intervals of the maximum varying points of the pulse waves is 10% or less of the heartbeat rate PRF at Step S320. When the determination at Step S320 is affirmed, the heartbeat rate PRF by subjecting the pulse wave signals to the FFT process is set as a detection heartbeat rate HR(i) at Step S340. This process then ends. When the determination at Step S320 is negated, an analysis result HR(i−1) is set as a detection heartbeat rate HR(i) at Step S350. This process then ends. Here, the detection heartbeat rate HR(i) is transmitted to the state determining unit 13 and the display controlling unit 19, and stored in the data storing unit 15.

Next, in the blood pressure analyzing process, as shown in FIG. 7, at first, an occurrence time ECGRt(i) of an R-wave peak detected from the electrocardiograph signal analyzing process is detected at Step S400. An occurrence time. PULSEt(i) of the maximum varying point detected in the pulse wave signal analyzing process is detected at Step S410. A pulse wave transmission time PTT(i) is computed by subtracting the occurrence time ECGRt(i) from the occurrence time PULSEt(i) at Step S420.

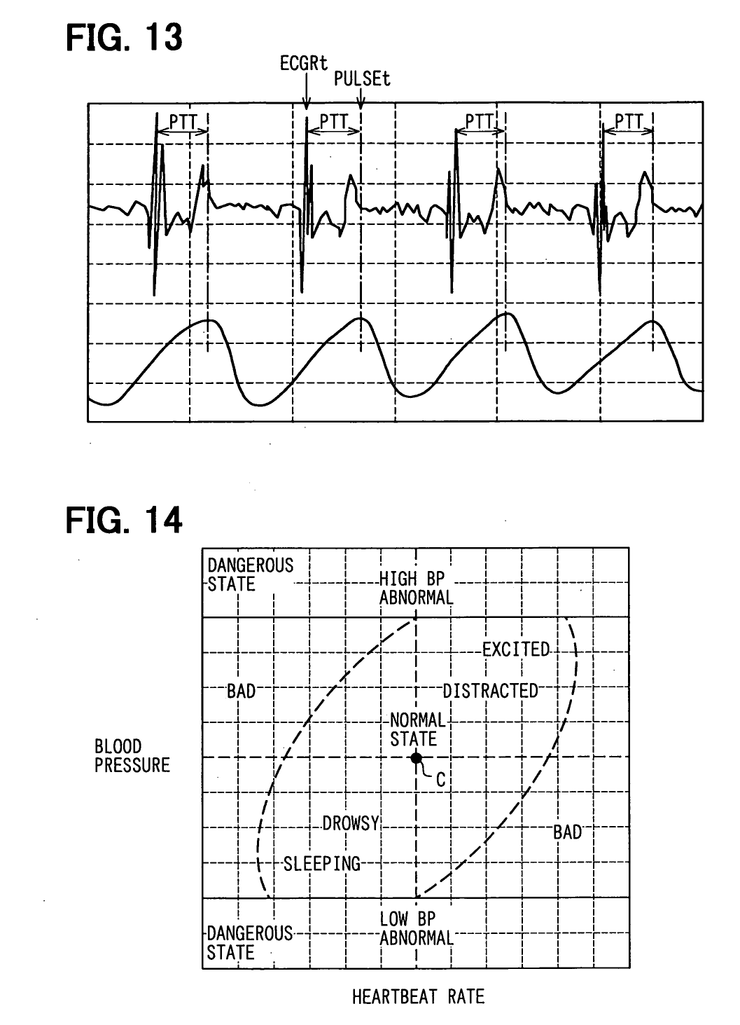

FIG. 13 shows a relationship among the occurrence times ECGRt, PULSEt, and the pulse wave transmission time PTT. Here, to make it easier to understand the drawing, an occurrence time of a peak is shown instead of the maximum varying point. Further, to compute the pulse wave transmission time PTT, either a maximum varying point or a peak of the pulse wave can be used. When the peak of the pulse wave is used instead of the maximum varying point, only a conversion coefficient for computing a blood pressure from the pulse wave transmission time PTT is differentiated.

At Step S430, an average value PTTav is computed for pulse wave transmission times PTT(i) computed for passed constant period (here, 60 seconds). Further, at Step S440 a detection blood pressure BP(i) is computed based on the pulse wave transmission time PTT(i) computed at Step S420, the pulse wave average time PPTav computed at Step S430, and an initial blood pressure BPr set at the display condition setting process that is to be explained later, using Equation (2).
BP(i)=(PTT(i)−PTTav)×(−0.5)+BPr (2)

Here, the detection blood pressure BP(i) obtained from this process is given to the state determining unit 13 and the display controlling unit 19, and stored in the data storing unit 15.

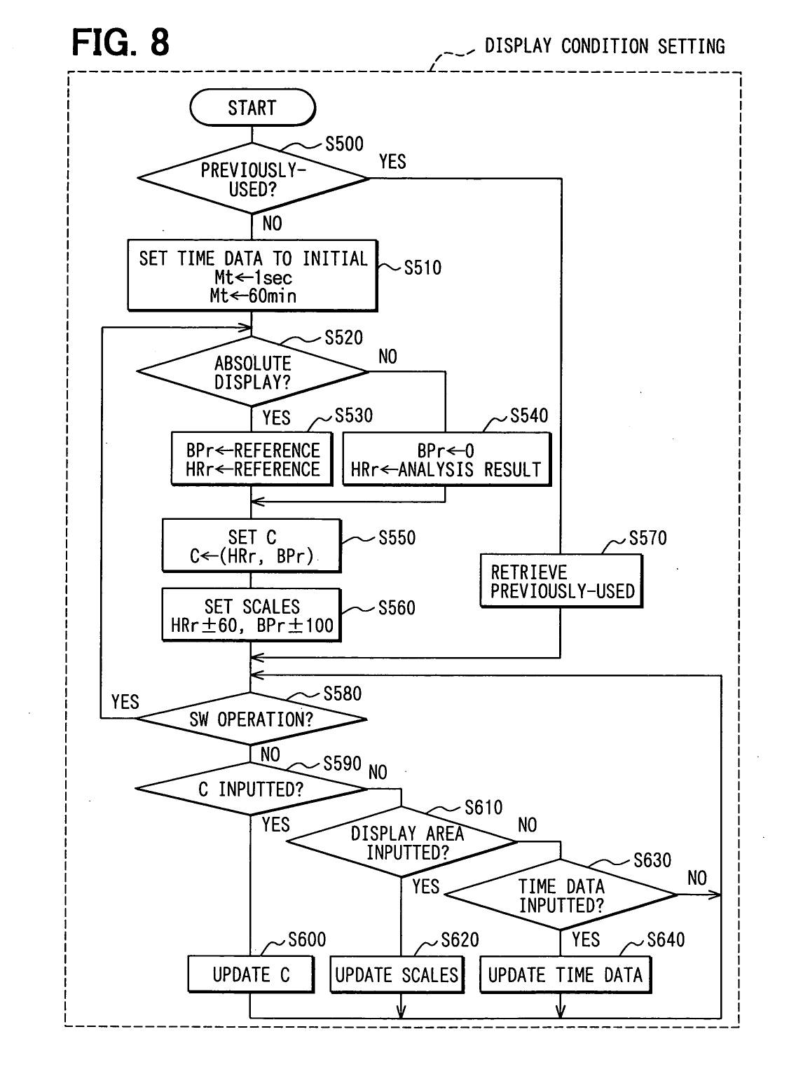

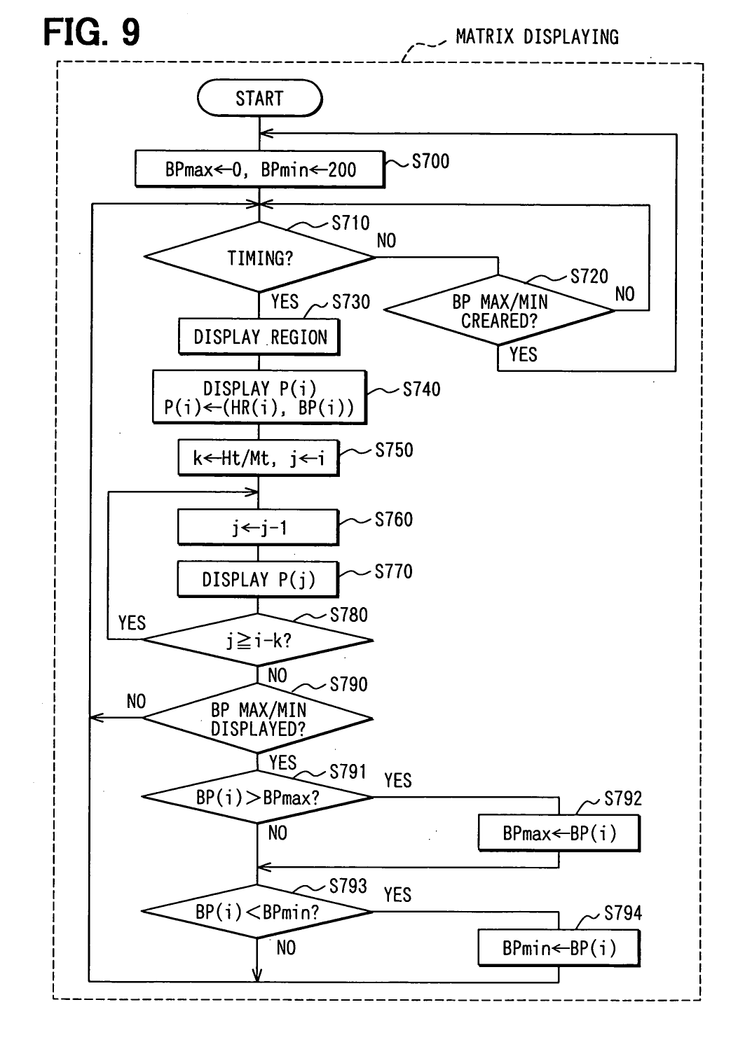

Next, the display condition setting process and the matrix displaying process both corresponding to the display controlling unit 19 will be explained with reference to flowcharts in FIGS. 8, 9. The display condition setting process starts when a blood pressure-heartbeat rate display is selected as display information in the input unit 3. The matrix displaying process starts when a matrix display is subsequently selected as a display method or form in the input unit 3.

In the display condition setting process, as shown in FIG. 8, at first, it is determined whether a previously-used value is set in the input unit 3 to be used currently (at the present timing) at Step S500. The previously-used value is a value that was used after the system 1 immediately-previously started. When it is not set, an initial setting takes place at Step S510. The initial setting sets time data including: a data update cycle Mt for updating a display to the latest data; and a record data display time Ht representing a holding time for holding displaying of passed data. Here, Mt is set to 1 second; Ht is set to 60 seconds.

Next, it is determined whether an absolute display is selected in the input unit 3 at Step S520. When an absolute display is selected, an initial blood pressure BPr is set as a predetermined blood pressure reference value while an initial heartbeat rate HRr is set as a predetermined heartbeat rate reference value at Step S530. Here, the blood pressure reference value and the heartbeat rate reference value can be predetermined to a typical average value, or to an average value corresponding to the user of the system 1 who is under resting conditions.

In contrast, when an absolute display is determined to be not selected at Step S520, when a relative display is selected, or when an absolute display and a relative display are not selected, an initial blood pressure BPr is set to zero and an initial heartbeat rate HRr is set based on an analysis result in the heartbeat rate analyzing process at Step S540 so as to start a relative display. In detail, this initial heartbeat rate HRr is an average of analysis results obtained for a predetermined constant time period (e.g., 60 seconds) after the system 1 starts.

Then, the initial blood pressure BPr and the initial heartbeat rate HRr are set as a value of an X-axis and a value of a Y-axis, respectively, in a two-dimensional coordinate system so that these values correspond to central coordinates C corresponding to the central position of the display screen of the displaying unit 5 at Step S550. Further, display scales of an X-axis and a Y-axis are set at Step S560 so that an area ranging between BPr±100 (mmHg) and HRr±60 (times) is shown within the display screen of the displaying unit 5 with the central coordinates being centered.

Namely, at Steps S510 to S560, as the display condition, the data update cycle Mt, the record data display time Ht, the initial blood pressure BPr, the initial heartbeat rate HRr, the central coordinates C, and the display scales are automatically set to initial values. Here, the values initially set are stored as the previously-used values in the data storing unit 15.

In contrast, when a previously-used value is set to be used currently at Step S500, the previously-used values are retrieved from the data storing unit 15 and an initial setting for the display condition takes place at Step S570.

After the initial setting, it is determined whether a display switching operation for switching between the absolute display and the relative display is conducted or not, at Step S580. When the display switching operation is conducted, the sequence returns to Step S520. Here, based on the display setting selected by the relevant operation, the initial blood pressure BPr, the initial heartbeat rate HRr, the central coordinates C, and the display scales are set again to initial values at Steps S520 to S560.

In contrast, when the display switching is determined to be not conducted at Step S580, subsequent determinations take place as shown in FIG. 8. Namely, at Step S590, it is determined whether the central coordinates C are inputted in the input unit 3. At Step S610, it is determined whether a display area of the blood pressure and the heartbeat rate that should be displayed within the display screen is inputted. At Step S630, it is determined whether time data Mt, Ht are inputted. All the determinations are negated, the sequence returns to Step S580.

In contrast, at Step S590, when it is determined that the central coordinates C are inputted in the input unit 3, the central coordinates C, the initial heartbeat rate HRr, and the initial blood pressure BPr are updated by values inputted at Step S600. The sequence then returns to Step S580. At Step S610, when it is determined that the display area of the blood pressure and the heartbeat rate that should be displayed within the display screen is inputted, display scales are updated at Step S620 so that the display area (maximum values and minimum values of the blood pressure and maximum values and minimum values of the heartbeat rate) inputted can be displayed within the display screen. At Step S630, when it is determined that time data Mt, Ht are inputted, the time data Mt, Ht are updated by values inputted at Step S640. The sequence then returns to Step S580. Here, when the display conditions are updated at Steps S600, S620, S640, the relevant previously-used values stored in the data storing unit 15 are updated, accordingly. Namely, in the display condition setting process, the initial setting of the display condition can be conducted using either the predetermined initial values or the previously-used values. Further, the values once set can be changed via the input unit 3.

Next, in the matrix displaying process, as shown in FIG. 9, at first, at Step S700, a maximum value BPmax and a minimum value BPmin of a blood pressure are initialized to 0 mmHg and 200 mmHg, respectively. Then, at Step S710, it is determined whether it is a data update timing based on the data update cycle Mt set or updated in the display condition setting process. Further, it is determined at Step S720 whether a maximum value or a minimum value of a blood pressure is cleared in the input unit 3. When both determinations at Steps S710, S720 are negated, the sequence returns to Step S710 and remains by repeating process at Steps S710, S720. When the determination at Step S720 is affirmed, the sequence returns to Step S700, where the maximum and the minimum values of a blood pressure are initialized again. Here, clearing the maximum and the minimum values of a blood pressure can be designed to be conducted at each clearing timing predetermined.

In contrast, when it is determined to be a data update timing at Step S710, a two-dimensional coordinate system is set on the display screen of the displaying unit 5 based on the central coordinates C and the display scales set or updated in the display condition setting process. Further, a regional display is conducted to indicate a correspondence relationship between individual points of the two-dimensional coordinate system and a driver's physical condition or state based on the two-dimensional coordinate system set at Step S730. In this regional display, as shown in FIG. 14, several regions are shown as follows. A region where a blood pressure exceeds the initial blood pressure BPr by more than a predetermined given value regardless of a heartbeat rate HR is a “dangerous (high blood pressure abnormality) region.” A region where a blood pressure underruns the initial blood pressure BPr by more than a predetermined given value regardless of a heartbeat rate HR is a “dangerous (low blood pressure abnormality) region.” A region centralized at the central coordinates C and shaped of an ellipse extending to the right upper and the left lower is a “normal state region.” The other region excluding the above-described regions is a “bad state region.” The individual regions are shown with region names. Further, in particular, within “normal state region,” the right upper portion has a sign of “excited” and “distracted” while the left lower portion has a sign of “drowsy” and “sleeping.” Here, only regions can be displayed without the region names.

Region setting shown in FIG. 14 is based on the following facts. Namely, with respect to a healthy subject, a blood pressure and a heartbeat rate are highly correlated with each other, so that a locus of detection data P(i) ascends to the right-upper and descends to the left-lower

In contrast, when a subject is ill or suffers sudden physical condition change or when a subject has a hypertension, a heart disease, or an autonomic imbalance, a locus is deviated from the tendency of the foregoing locus. Unlike the healthy subject, there is a tendency that the heartbeat rate and the blood pressure do not change at the same time, but the one of them changes earlier than the other and the other subsequently follows the changing of the one.

Further, when a driver as the subject is drowsy or sleeping, a blood pressure and a heartbeat rate decrease at the same time. When a driver is excited or distracted, a blood pressure and a heartbeat rate increase at the same time.

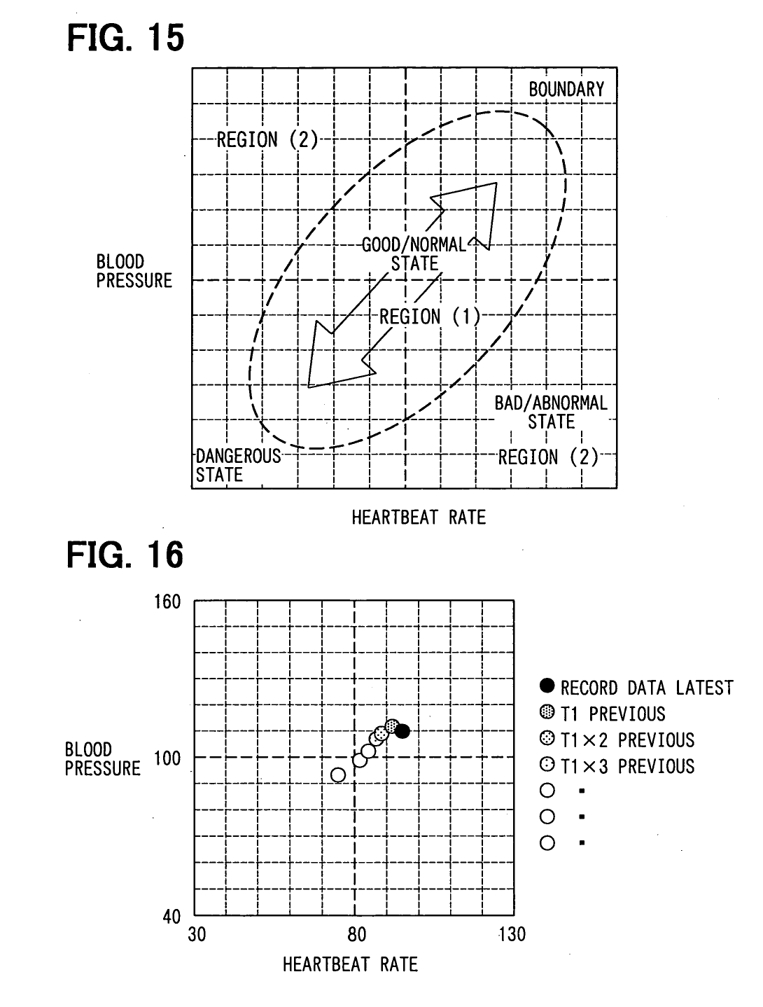

In other words, superimposing the locus of detection data P(i) on the regional display enables determination of whether the physical condition is in a normal state (Region (1) in FIG. 15) or a bad (or disordered)/abnormal state (Region (2) in FIG. 15). Further, this enables, within the normal state, determination of any one of a “relatively stable state,” an “excited state,” a “distracted state,” a “drowsy state,” or a “sleeping state.”

Next, in this two-dimensional coordinate system including the regional display, the detection data P(i) is shown while a heartbeat rate HR(i) of an analysis result from the heartbeat rate analyzing process being set to an X-axis while a blood pressure BP(i) of an analysis result from the blood pressure analyzing process being set to a Y-axis. The detection data P(i) is stored in the data storing unit 15 at Step S740.

Here, a heartbeat rate HR(i) and a blood pressure BP(i) use the latest analysis result (detection data) without change, in the heartbeat rate analyzing process and the blood pressure analyzing process, respectively, when a data update cycle Mt is not more than an analyzing cycle (1 second) of the electrocardiograph signal and the pulse wave signal.

In contrast, a heartbeat rate HR(i) and a blood pressure BP(i) use averages of detection data obtained within the data update cycle Mt when a data update cycle Mt is more than an analyzing cycle (1 second) of the electrocardiograph signal and the pulse wave signal.

Dividing the record data display time Ht by the data update cycle Mt provides a number k of record data P(j) that are displayed on the two-dimensional coordinate system. A variable i that designates the heartbeat rate HR(i) and blood pressure(i) displayed at Step S740 is substituted for a variable j at Step S750. The variable j is incremented by one at Step S760. A heartbeat rate HR(j) and a blood pressure BP(j) that are designated by the variable j constitute an X coordinate and a Y coordinate to form a record data P(j). This record data P(j) is displayed on the two-dimensional coordinate system with a color changing a tone (here, becoming brighter) by 1/k tone compared to that of data P(j+1) at Step S770.

Next, it is determined whether the variable j is (i−k) or more at Step S780. When this determination is affirmed, the sequence returns to Step S760, where the process repeats displaying the record data P(j). In contrast, when the determination is negated, namely, when k items of record data P(i−1) to P(i−k) are all displayed, the sequence goes to Step S790. Here, it is determined whether a maximum/minimum display setting is commanded by using the input unit 3. This maximum/minimum display setting enables displaying a maximum blood pressure value BPmax and a minimum blood pressure value BPmin.

When a maximum/minimum display setting is not conducted, the sequence returns to Step S710. When a maximum/minimum display setting is conducted, it is determined whether a blood pressure BP(i) is more than the maximum blood pressure value BPmax at Step S791. When this determination is affirmed, the maximum blood pressure BPmax is updated by the blood pressure BP(i) and the display of the maximum blood pressure value BPmax on the two-dimensional coordinate system is updated; further, the maximum blood pressure value BPmax and its detection time are stored in the data storing unit 15 at Step S792.

Subsequently, it is determined whether a blood pressure BP(i) is less than the minimum blood pressure value BPmin at Step S793. When this determination is affirmed, the minimum blood pressure BPmin is updated by the blood pressure BP(i) and the display of the minimum blood pressure value BPmin on the two-dimensional coordinate system is updated; further, the minimum blood pressure value BPmin and its detection time are stored in the data storing unit 15 at Step S794. The sequence then returns to Step S710.

In sum, in the matrix displaying process, through the process at Steps S730 to S780, as shown in FIGS. 16 to 18, on the two-dimensional coordinate system set on the display screen of the displaying unit 5, detection data P(i) and k items of record data P(i−1) to P(i−k) are shown with the regional display. Here, k items of record data P(i−1) to P(i−k) are displayed so that the color tone of the record data becomes lower as record data becomes older. Displaying of the maximum and minimum blood pressure values BPmax, BPmin can be arbitrarily selected between displaying and not-displaying. Further, displaying of the maximum and minimum blood pressure values BPmax, BPmin can be arbitrarily cleared. Here, in FIGS. 16 to 18, to easily understand the drawings, the regional display is removed.

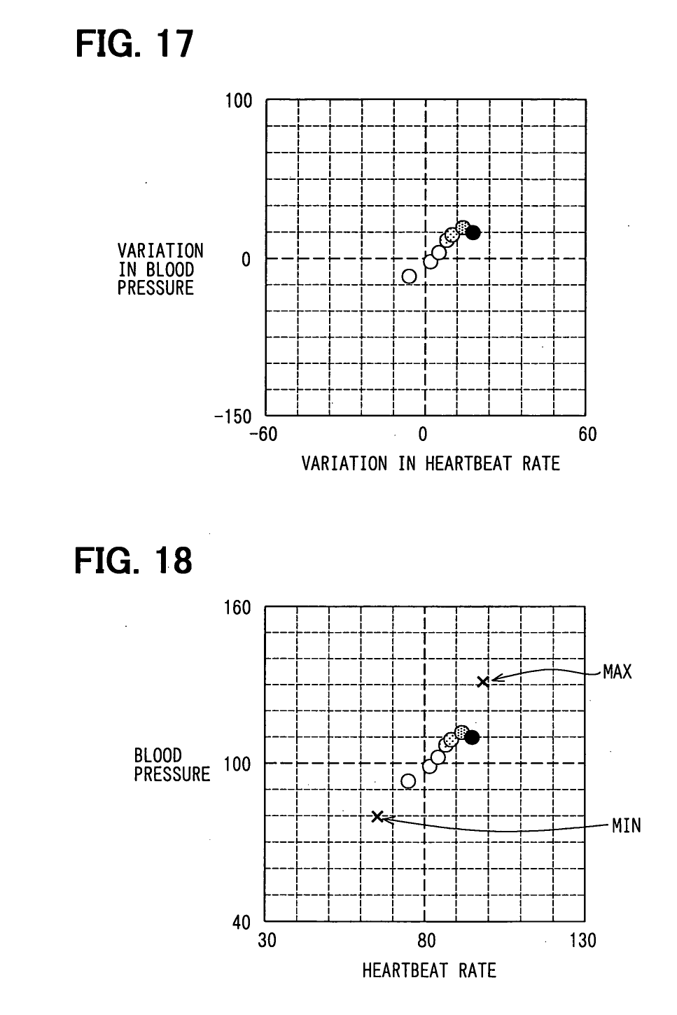

FIG. 16 shows an absolute display where the central coordinates C is (80, 100), namely an initial heartbeat rate HRr is 80 and an initial blood pressure BPr is 100 (mmHg). Here, display scales are HRr±50 in the X-axis and BPr±60 in the Y-axis. FIG. 17 shows a relative display where the central coordinates C is (0, 0), and display scales are HRr±50 in the X-axis and BPr±60 in the Y-axis similarly.

FIG. 18 shows a display that is shown through the process at Steps S791 to S794 when maximum/minimum values are set to be displayed. The maximum/minimum values are represented by “x” to be distinguished from detection data or record data that are represented by “O.”

Here, the maximum value or the minimum value functions as a characteristic value to represent a characteristic of the living body information within a given time interval based on detection results. Further, an average value can also work as a characteristic value. These characteristic values are associated with points on the two-dimentional coordinate system on the display screen while being distinguished from detection results. Thus, using the characteristic value enables proper understanding of whether a variation trend of a subject is transient or chronical.

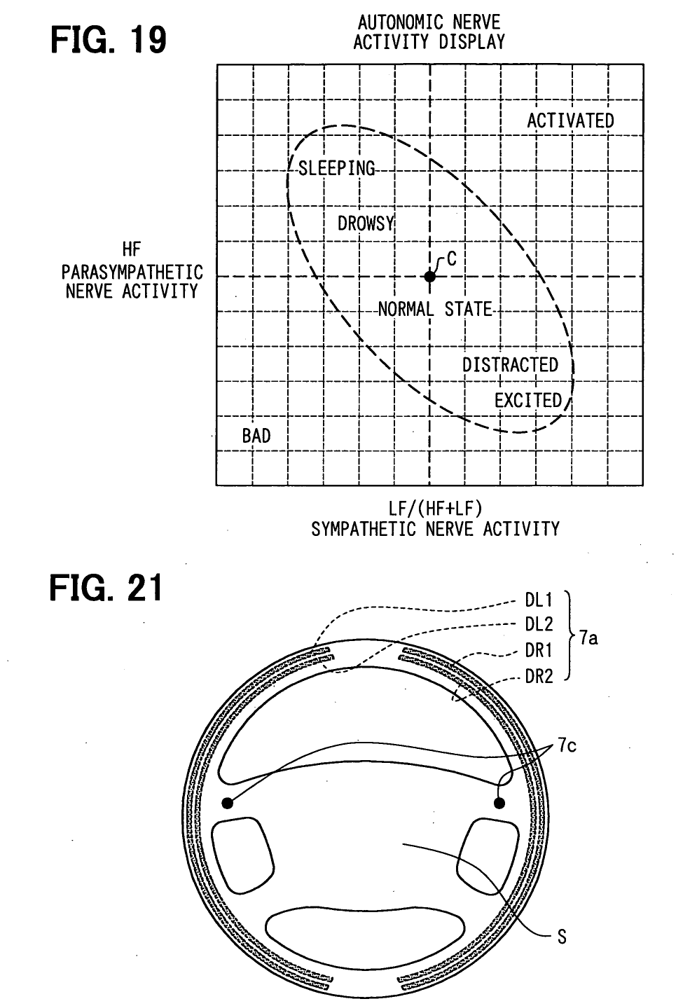

In the above embodiment, a matrix display is explained that displays a pair of a blood pressure and a heartbeat rate as living body information. Further, the matrix display can display an autonomic nerve activity as living body information. In this case, a sympathetic nerve activity is set to an X-axis while a parasympathetic nerve activity is set to a Y-axis. In this regional display shown in FIG. 19, a region that is centralized at the central coordinates C and shaped of an ellipse extending to the left upper and the right lower is “normal state region.” Within regions other than “normal state region,” a region located in the right upper is “physically active region”; a region located in the left lower is “physically bad (or disordered) region.” Further, within “normal state,” the left upper portion has a sign of “drowsy” and “sleeping”; the right lower portion has a sign of “excited” and “distracted.” The regional display can remove the signs or names instead.

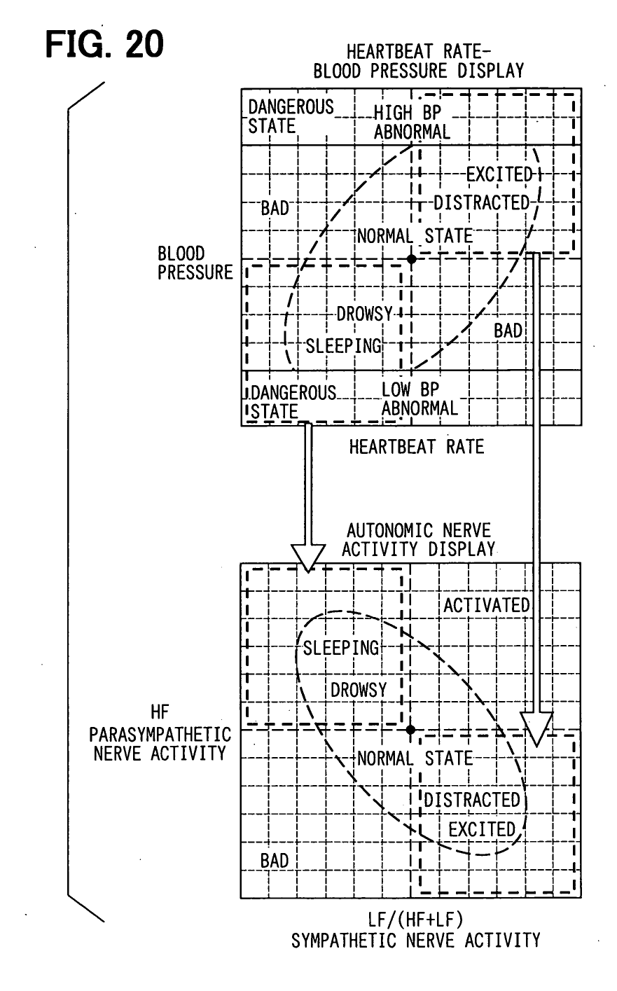

Compared to a matrix display of a pair of a blood pressure and a heartbeat rate, as shown in FIG. 20, in general, the first quadrant and the third quadrant in the blood pressure-heartbeat rate display correspond to the fourth quadrant and the second quadrant in the autonomic nerve activity display, respectively.

In the autonomic nerve activity display, a locus of detection data P(i) with respect to a normal healthy subject ascends to the left and descends to the right. With respect to a sick subject or a physically disordered subject, a locus of detection data deviates from the foregoing locus.

An autonomic nerve activity display and a blood pressure-heartbeat rate display are used in common and switched therebetween. This enables recognition of whether an increase in a heartbeat rate or blood pressure is caused by a result of sympathetic nerve activity or others.

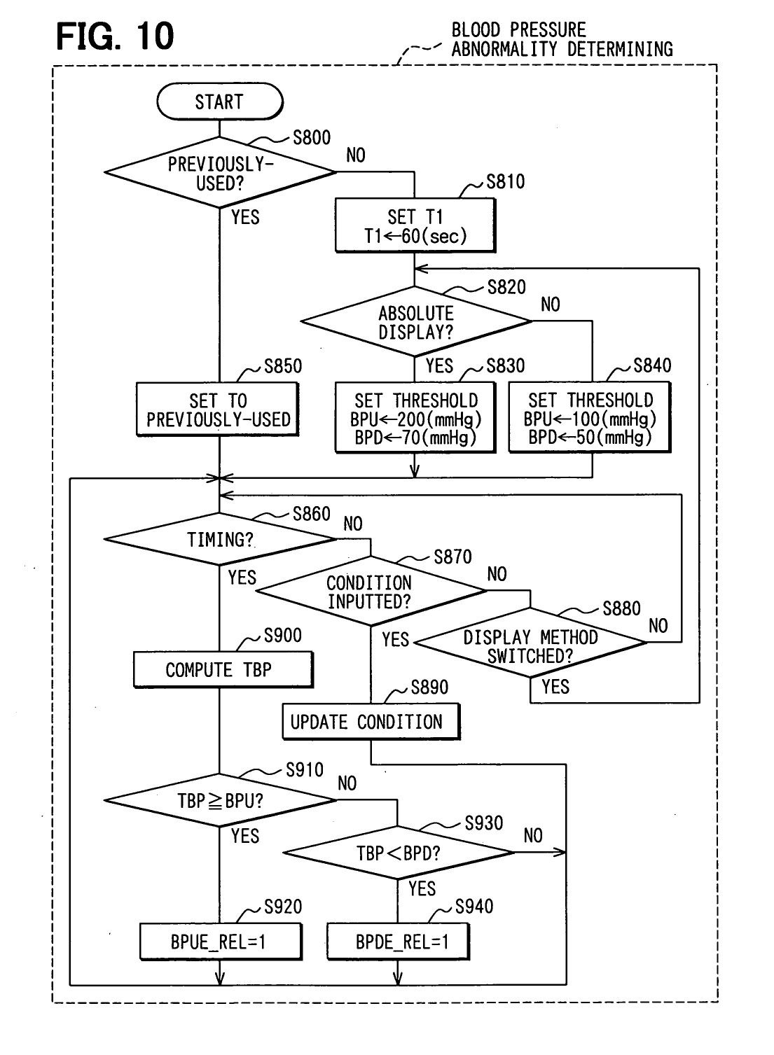

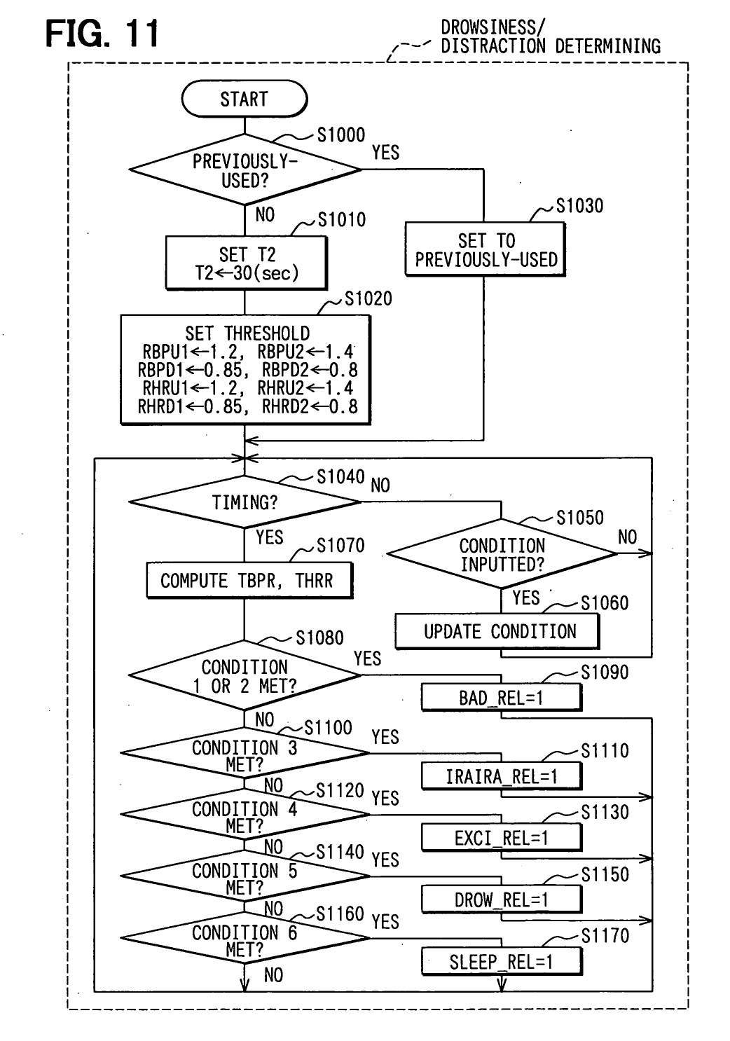

Next, the blood pressure abnormality determining process and the drowsiness/distraction determining process both corresponding to the state determining unit 13 will be explained with reference to flowcharts in FIGS. 10, 11. Both the processes start when electric power is supplied to the system 1.

In the blood pressure abnormality determining process, as shown in FIG. 10, at first, it is determined whether a previously-used value is set to be currently used as a blood pressure abnormality determination condition at Step S800. The previously-used value is a value that was used after the system 1 immediately-previously started. When it is not set, a determination cycle T1 for executing a determination of blood pressure abnormality is set to an initial value (in this embodiment, 60 seconds) at Step S810. The determination cycle T1 is preferably set to a value significantly larger (e.g., 10 times or more) than the data update cycle Mt in order to obtain a stable determination result.

At Step S820, it is determined whether an absolute display is selected by the input unit 3. When an absolute display is selected, a high blood pressure threshold value BPU for determining blood pressure abnormality is set to a given predetermined value (in this embodiment, 200 mmHg) while a low blood pressure threshold value BPD is set to a given predetermined value (in this embodiment, 70 mmHg) at Step S830.

In contrast, at Step S820, when it is determined that an absolute display is determined to be not selected, when a relative display is selected, or when an absolute display and a relative display are not selected, a maximum blood pressure threshold value BPU is set to a given predetermined value (in this embodiment, 100 mmHg) and a minimum blood pressure threshold value BPD is set to a given predetermined value (in this embodiment, −50 mmHg) at Step S840.

In sum, through the process at Steps S810 to S840 an initial setting as a blood pressure abnormality determination condition is automatically applied to a blood pressure abnormality determination cycle T1, a high blood pressure threshold value BPU, and a low blood pressure threshold value BPD. The values thus set are stored as previously-used values in the data storing unit 15.

In contrast, when a previously-used value is set to be currently used at Step S800, the previously-used values are retrieved from the data storing unit 15 and an initial setting for the blood pressure abnormality determination condition takes place at Step S850.

After the initial setting, it is determined whether it is a determination timing for determining blood pressure abnormality based on a blood pressure abnormality determination cycle T1 at Step S860. It is then determined at Step S870 whether a blood pressure abnormality determination condition (blood pressure abnormality determination cycle T1, high blood pressure threshold value BPU, and low blood pressure threshold value BPU) is inputted in the input unit 3. It is then determined at Step S880 whether a display method is switched between an absolute display and a relative display in the input unit 3. When all the foregoing determinations at Steps S860, S870, S880 are negated, the sequence returns to Step S860, where the sequence waits ready by repeating the process at Steps S860 to S880.

At Step S880, when the determination is affirmed, the sequence returns to Step S820, where a high blood pressure threshold value BPU, and a low blood pressure threshold value BPD are set again based on the display method set by an operation in the input unit 3.

At Step S870, when the determination is affirmed, the blood pressure abnormality determination condition is updated based on a value inputted at Step S890. The sequence then returns to Step S860. Here, each time the abnormality determination condition is updated at Step S890, the previously-used values stored in the data storing unit 15 are updated similary. Further, in particular, when a high blood pressure threshold value BPU or a low blood pressure threshold value BPD is updated, “dangerous (high blood pressure abnormality) region” or “dangerous (low blood pressure abnormality) region” in the regional display can be modified in borders.

When a determination at S860 is affirmed, namely when it is a timing for determining blood pressure abnormality, a blood pressure average value TBP is computed as an average of blood pressures BP(i) computed during the the blood pressure abnormality determination cycle T1 at Step S900. It is then determined at Step S910 whether the blood pressure average TBP is the high blood pressure threshold value BPU or more. It is then determined whether the blood pressure average TBP is the low threshold value BPD or less at Step S930. When both the determinations at Steps S910, S930 are negated, the sequence returns to Step S860.

When the determination at Step S910 is affirmed, the driver is regarded as being in a high blood pressure abnormal state. A high blood pressure abnormality flag BPUE_REL is set to 1 at Step S920. When the determination at Step S930 is affirmed, the driver is regarded as being in a low blood pressure abnormal state. A low blood pressure abnormality flag BPDE_REL is set to 1 at Step S940. The sequence then returns to Step S860. When abnormality flags are set at Steps S920, S930, the kind of the relevant abnormality flag and its detection time are stored in the data storing unit 15.

In sum, in the blood pressure abnormality determining process, blood pressure abnormality is determined every a blood pressure abnormality determination cycle T1. The initial setting for the blood pressure abnormality determination condition can be conducted using the predetermined initial values or the previously-used values. Further, the blood pressure abnormality determination condition once set can be arbitrarily changed via the input unit 3.

In the drowsiness/distraction determining process, as shown in FIG. 11, at first, it is determined whether a previously-used value is set to be currently used as a drowsiness/distraction determination condition at Step S1000. The previously-used value is a value that was used after the system 1 immediately-previously started. When it is not set, a determination cycle T2 for executing a determination of drowsiness/distraction is set to an initial value (in this embodiment, 30 seconds).

At Step S1020, various values for the drowsiness/distraction determination are set as follows. A first upper blood pressure ratio threshold value RBPU1 is set to a predetermined value (in this embodiment, 1.2); a second upper blood pressure ratio threshold value RBPU2 is set to a predetermined value (in this embodiment, 1.4); a first lower blood pressure ratio threshold value RBPD1 is set to a predetermined value (in this embodiment, 0.85); a second lower blood pressure ratio threshold value RBPD2 is set to a predetermined value (in this embodiment, 0.8); a first upper heartbeat rate ratio threshold value RHRU1 is set to a predetermined value (in this embodiment, 1.2); a second upper heartbeat rate ratio threshold value RHRU2 is set to a predetermined value (in this embodiment, 1.4); a first lower heartbeat rate ratio threshold value RHRD1 is set to a predetermined value (in this embodiment, 0.85); and a second lower heartbeat rate ratio threshold value RHRD2 is set to a predetermined value (in this embodiment, 0.8).

In sum, through the process at Steps S1010 to S1020, as a drowsiness/distraction determination condition, initial setting is automatically applied to the drowsiness/distraction determination cycle T2, and threshold values RBPU1, RBPU2, RBPD1, RBPD2, RHRU1, RHRU2, RHRD1, RHRD2. These set values are stored as the previously-used values in the data storing unit 15.

In contrast, when a previously-used value is set to be currently used at Step S1000, the previously-used values are retrieved from the data storing unit 15 and an initial setting for the drowsiness/distraction determination condition takes place at Step S1030.

After the initial setting, it is determined whether it is a determination timing for determining drowsiness/distraction based on a drowsiness/distraction determination cycle T2 at Step S1040. It is then determined at Step S1050 whether a drowsiness/distraction determination condition is inputted in the input unit 3. When both the foregoing determinations at Steps S1040, S1050 are negated, the sequence returns to Step S1040, where the sequence waits ready by repeating the process at Steps S1040 to S1050.

At Step S1050, when the determination is affirmed, the sequence goes to Step S1060, where a drowsiness/distraction determination condition is updated based on a value inputted at Step S1060. The sequence then returns to Step S1040. Here, each time the determination condition is updated at Step S1060, the previously-used values stored in the data storing unit 15 are updated similarly.

When a determination at S1040 is affirmed, namely when it is a timing for determining drowsiness/distraction, a blood pressure average ratio TBPR and a heartbeat rate average ratio THRR are computed with respect to a drowsiness/distraction determination cycle T2 at Step S1070. Here, the blood pressure average ratio TBPR is obtained from Equation (3) when a display method is selected as an absolute display or obtained from Equation (4) when a display method is selected as a relative display. In contrast, the heartbeat rate average ratio THRR is obtained from Equation (5) regardless of display methods.

$$\begin{matrix}TBPR = \frac{{\sum }_{i}^{n} BP ( i )}{n} / BPr & ( 3 ) \\ TBPR = \frac{{\sum }_{i}^{n} ( PTTav - PTT ( i ) )}{PTTav / n} & ( 4 ) \\ THRR = \frac{{\sum }_{i}^{n} HR ( i )}{n} / HRr & ( 5 )\end{matrix}$$

Here, “n” is the number of blood pressures BP(i) and heartbeat rates HR(i) that are obtained during the determination cycle T2.

Then, at Step S1080 it is determined whether the blood pressure average ratio TBPR and the heartbeat rate average ratio THRR computed at Step S1070 meet a condition 1 or a condition 2. The condition 1 is that the blood pressure average ratio TBPR is the first upper blood pressure ratio threshold value RBPU1 or more and the heartbeat rate average ratio THRR is the first lower heartbeat rate ratio threshold value RBRD1 or less (i.e., TBPR≧RBPU1 and THRR≦RHRD1). The condition 2 is that the blood pressure average ratio TBPR is the first lower blood pressure ratio threshold value RBPD1 or less and the heartbeat rate average ratio THRR is the first upper heartbeat rate ratio threshold value RBRU1 or more (i.e., TBPR≦RBPD1 and THRR≧RHRU1). The determination at Step S1080 is affirmed, it is determined that a physical condition is disordered and a flag of bad health BAD_REL is set to 1 at Step S1090. The sequence returns to Step S1040.

When the blood pressure average ratio TBPR and the heartbeat rate average ratio THRR computed at Step S1070 are determined not to meet the condition 1 or the condition 2 at Step S1080, it is then determined whether the blood pressure average ratio TBPR and the heartbeat rate average ratio THRR meet a condition 3 at Step S100. The condition 3 is that the blood pressure average ratio TBPR is not less than the first upper blood pressure ratio threshold value RBPU1 and not more than the second upper blood pressure ratio threshold value RBPU2, and the heartbeat rate average ratio THRR is not less than the first upper heartbeat rate ratio threshold value RBRU1 or not more than the second upper heartbeat rate ratio threshold value RBRU2 (i.e., RBPU1≦TBPR≦RBPU2 and RBPU1≦THRR≦RHRU2). The determination at Step S1100 is affirmed, it is determined that a physical condition is distracted (IRAIRA in the Japanese language) and a flag of being distracted IRAIRA_REL is set to 1 at Step S110. The sequence then returns to Step S1040.

When the blood pressure average ratio TBPR and the heartbeat rate average ratio THRR are determined not to meet the condition 3 at Step S1100, it is then determined whether the blood pressure average ratio TBPR and the rate average ratio THRR meet a condition 4 at Step S1120. The condition 4 is that the blood pressure average ratio TBPR is more than the second upper blood pressure ratio threshold value RBPU2 and the heartbeat rate average ratio THRR is more than the second upper heartbeat rate ratio threshold value RBRU2 (i.e., TBPR>RBPU2 and THRR>RHRU2). The determination at Step S1120 is affirmed, it is determined that a physical condition is excited and a flag of being excited EXCI_REL is set to 1 at Step S1130. The sequence then returns to Step S1040.

When the blood pressure average ratio TBPR and the heartbeat rate average ratio THRR are determined not to meet the condition 4 at Step S1120, it is then determined whether the blood pressure average ratio TBPR and the rate average ratio THRR meet a condition 5 at Step S1140. The condition 5 is that the blood pressure average ratio TBPR is not less than the second lower blood pressure ratio threshold value RBPD2 and not more than the first lower blood pressure ratio threshold value RBPD1 the heartbeat rate average ratio THRR is not less than the second lower heartbeat rate ratio threshold value RBRD2 and not more than the first lower heartbeat rate ratio threshold value RBRD1 (i.e., RBPD2≦TBPR≦RBPD1 and RHRD2≦THRR≦RHRD1). The determination at Step S1140 is affirmed, it is determined that a physical condition is drowsy and a flag of being drowsy DROW_REL is set to 1 at Step S1150. The sequence then returns to Step S1040.

When the blood pressure average ratio TBPR and the heartbeat rate average ratio THRR are determined not to meet the condition 5 at Step S1140, it is then determined whether the blood pressure average ratio TBPR and the rate average ratio THRR meet a condition 6 at Step S1160. The condition 6 is that the blood pressure average ratio TBPR is less than the second lower blood pressure ratio threshold value RBPD2 and the heartbeat rate average ratio THRR is less than the second lower heartbeat rate ratio threshold value RBRD2 (i.e., TBPR<RBPD2 and THRR<RHRD2). The determination at Step S1160 is affirmed, it is determined that a physical condition is sleeping and a flag of being sleeping SLEE_REL is set to 1 at Step S1170. The sequence then returns to Step S1040.

When the blood pressure average ratio TBPR and the heartbeat rate average ratio THRR are determined not to meet the condition 6 at Step S1160, the sequence then returns to Step S1040. In other words, the drowsiness/distraction determining process determines one of the various physical conditions of the bad condition, the distracted condition, the excited condition, the drowsy condition, and the sleeping condition, every a drowsiness/distraction determination cycle T2. The initial setting of the drowsiness/distraction determination condition can use either the predetermined initial values or the previously-used values. Further, the initial values that are once set can be arbitrarily changed via the input unit 3.

In this embodiment, the determination is conducted based on the blood pressure and the heartbeat rate; however, it can be conducted based on the autonomic nerve activity or a combination of the blood pressure, the heartbeat rate, and the autonomic nerve activity. In detail, for instance, suppose a case that the coordinates of a detection data P(i) is located within the first quadrant in the blood pressure-heartbeat rate display and significantly deviated from the fourth quadrant in the autonomic nerve activity display. Further, suppose another case that the coordinates of a detection data P(i) is located within the third quadrant in the blood pressure-heartbeat rate display and significantly deviated from the second quadrant in the autonomic nerve activity display. In these two cases, it can be determined that the physical condition is disordered.

Determination results in the state determining unit 13 (e.g., high blood pressure abnormality determining process and drowsiness/distraction determining process) are provided to the actuation unit 17 as various flags. The various flags include the high blood pressure abnormality flag BPUE_REL, the low blood pressure abnormality flag BPDE_REL, the bad health flag BAD_REL, the distraction flag IRAIRA_REL, the excitement flag EXCI_REL, the drowsiness flag DROW_REL, and the sleeping flag SLEE_REL. The actuation unit 17 conducts a control corresponding to an abnormality or a state designated by each of the determination results.

In detail, when it is determined that the physical condition is in a bad health (BAD_REL=1), the following takes place: a warning display or sound that indicates the bad health; automatic notification of the present position to a previously designated contact point; a display of the contact point; or a guiding display for indicating a route to a neighboring medical institution or a place where the vehicle can be parked.

Further, when the distracted state (IRAIRA_REL=1) or the excited state (EXCI_REL=1) is determined, reproduction of a music composition having an effect of relaxing the distracted or excited state can be achieved in addition to the warning display or the warning sound.

Further, when the drowsy state (DROW_REL=1) or the sleeping state (SLEE_REL=1) is determined, reproduction of a music composition having an effect of elevating the driver who is drowsy or sleeping or various control for wakening the driver can be achieved in addition to the warning display or the warning sound. The various controls include opening of a window, changing of a temperature, a wind direction, or a wind power.

In the embodiment of the system 1, the electrodes detect the electrocardiograph signals or the pulse signals using the electrocardiograph sensor 7a and the optical pulse wave sensor 7b provided in the steering wheel S. Then, these obtained electrocardiograph signals or pulse wave signals are used for obtaining body information (blood pressure, heartbeat rate, or sympathetic nerve activity). Therefore, continuous acquirement of body information neither needs physical burdens of the driver nor prevents driving by the driver.

In the embodiment of the system 1, detection data P(i) of two kinds of body information continuously obtained are associated with points in the two-dimensional coordinate system. The record data P(i−1)˜P(i−k) are shown in the display screen along with the regional display showing a correspondence relationship between points in the two-dimensional coordinate system and a driver's state.

Therefore, the detection data P(i) shown in the two-dimensional coordinate system along with the regional display can simply indicate the conditions or states of the driver without needing any professional medical knowledge. Further, the detection data P(i) along with the record data P(i−1)˜P(i−k) can also indicate the present states and variations in states in real time.

Further, in the embodiment of the system 1, the analysis results HR(i), BP(i) are used for determining the conditions or states of the driver. Therefore, even when the driver misses seeing the display of the detection data P(i), proper action can be conducted based on the determination results.

Further, in the embodiment of the system 1, the various items can be selected. Here, the various items include body information to be displayed, display methods or forms of body information (matrix display or trend display, absolute display or relative display), displaying or not displaying of maximum and minimum values, or setting methods of initial values for display conditions or determination conditions. The display conditions or the determination conditions can be arbitrarily selected and customized by the driver for display or determination suitable for individual drivers.

In this embodiment, the signal measuring unit 7 functions as a detecting unit for body information; the process at Steps S740 to S780 functions as a detection result displaying unit; the process at Step S730 functions as a region displaying unit, the process at Steps S791 to S794 functions as a characteristic value computing unit; the input unit 3 functions as a designating unit for a kind of body information; the process at Steps S590 to S640 functions as a display condition changing unit; the state determining unit 13 functions as a determining unit for a state of a subject; and the actuation unit 17 functions as an executing unit.

(Others)

In the embodiment, a portable wrist-watch-shaped pulse wave sensor is adopted as the pulse wave sensor 7b. However, as shown in FIG. 21, a pulse wave sensor can be a stational-typed pulse wave sensor 7c that is embedded in places which a driver touches within the steering wheel S.

In the embodiment, the present invention is directed to a physical condition monitor system mounted in a vehicle. However, the present invention can be also directed to a medical-purposed measuring unit in medical sites. In this case, the signal measuring unit 7 can include a catheter remaining within an artery, a blood oxygen level meter SPO2, or an electrocardiography, a periodic blood pressure meter (korotokov, oscillometric method).

In the embodiment, color tones of the record data plotted in the two-dimensional coordinate system are step-wise changed to indicate chronological order at a glance. However, shapes or sizes of the record data plotted can be changed instead.

In the embodiment, other than the detection data or the record data, the maximum and minimum points of the blood pressure are shown; however, an average during a predetermined time interval can be displayed instead.

In the embodiment, the state determining unit 13 determines conditions or states by comparing with various threshold values; however, time-wise variation can be also used for determining. In other words, conflict with drowsiness can be detected by movement advancing and returning between a lower region and a normal region for a given period.

In the embodiment, body information includes a blood pressure, a heartbeat rate, or an autonomic nerve activity. However, abnormal cardiac rhythms detected from electrocardiograph signals can be used in combination with other body information for specifically determining the physical abnormality.

In the embodiment, the initial data uses predetermined initial values or previously-used vales in the display condition of the matrix displaying process and in the determination condition of the blood pressure abnormality determining process or the drowsiness/distraction determining process. However, the initial values can include values based on data obtained for a given period from driving start or re-start or based on an average of data obtained and accumulated at multiple normal driving starts.

It will be obvious to those skilled in the art that various changes may be made in the above-described embodiments of the present invention. However, the scope of the present invention should be determined by the following claims.

What is claimed is:

1. A living body information displaying method comprising steps of:
associating detection results of two kinds of living body information, which are obtained with respect to a subject, with a point on a two-dimensional coordinate system that is set on a display screen; and
displaying on the display screen
the detection results along with previously obtained detection results of the two kinds of living body information and
a region which indicates a correspondence relationship between a point on the two-dimensional coordinate system and a state of the subject.

2. A living body information displaying device comprising:
a detecting unit that obtains detection results of at least two kinds of living body information based on a pulse wave signal and an electrocardiograph signal of a subject;
a detection result displaying unit that associates the detection results with a point on a two-dimensional coordinate system that is set on a display screen to thereby display the detection results along with previously obtained detection results of the two kinds of living body information on the display screen; and
a region displaying unit that displays on the display screen a plurality of regions that indicate correspondence relationships between points of the two-dimensional coordinate system and states of the subject to overlap the regions to detection results displayed by the detection result displaying unit.

3. The living body information displaying device of claim 2,
wherein the previously obtained detection results are displayed in a display form that enables recognition of a time series of the previously obtained detection results.

4. The living body information displaying device of claim 2, further comprising:
a characteristic value computing unit that computes a characteristic value that represents a characteristic of living body information within a given time interval based on detection results obtained by the detecting unit,
wherein the detection result displaying unit displays the characteristic value that is associated with a point on the two-dimensional coordinate system in a display form that enables the characteristic value to be distinguished from the detection results obtained by the detecting unit.

5. The living body information displaying device of claim 2, further comprising:
a designating unit that designates a kind of living body information that is displayed by the detection result displaying unit,
wherein the region displaying unit displays a region corresponding to the kind of living body information designated.

6. The living body information displaying device of claim 2,
wherein the detecting unit detects at least a heartbeat rate and a blood pressure as the at least two kinds of living body information.

7. The living body information displaying device of claim 2,
wherein the detecting unit detects at least a sympathetic nerve activity amount and a parasympathetic nerve activity amount as the at least two kinds of living body information.

8. The living body information displaying device of claim 2, further comprising:
a display condition changing unit that changes
a scale of a coordinate axis of the two-dimensional coordinate system set on the display screen and
a set of coordinates that is a center of the display screen.

9. The living body information displaying device of claim 2, further comprising:
a determining unit that determines a state of the subject based on detection results obtained by the detecting unit; and
an executing unit that executes a control for assisting an action of the subject or a control for improving the state of the subject, based on the state of the subject determined.
